(19) United States
(12) Patent Application Publication
(10) Pub. No.: US 2009/0030233 A1
(43) Pub. Date: Jan. 29, 2009

(54) PROCESS FOR THE PRODUCTION OF BIPHENYLS

(75) Inventors: Josef Ehrenfreund, Allschwil (CH); Harald Walter, Stein (CH); Hans Tobler, Basel (CH); Camilla Corsi, Stein (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA CROP PROTECTION, INC., Greensboro, NC (US)

(21) Appl. No.: 12/092,324

(22) PCT Filed: Nov. 13, 2006
(86) PCT No.: PCT/EP2006/010864

§ 371 Date: Aug. 22, 2008

(30) Foreign Application Priority Data
Nov. 15, 2005 (EP) 05024967.1

(51) Int. Cl. C07C 211/45; C07C 205/06

(52) U.S. Cl. 564/305; 568/928

(57) ABSTRACT

The present invention relates to a novel process for the preparation of a compound of formula (I): which comprises reacting a compound of formula (II): with a compound of general formula (III): wherein R¹ and R² are H or C₁₋₄ alkyl or R¹ and R² join together to form a C₂₋₃ alkylene group, which is optionally substituted by from 1 to 4 methyl or ethyl groups, or an anhydride of the compound (III), in the presence of a base and of a palladium catalyst which is (a) a palladium (0)- or palladium (II)-triarylphosphine complex optionally in the presence of additional amounts of a triarylphoshine ligand, or (b) a palladium (II) salt in the presence of a triarylphosphine ligand, or (c) metallic palladium, optionally deposited on a support, in the presence of triarylphosphine; there being used from 0.9 to 2 moles of compound (III) for each mole of compound (II).

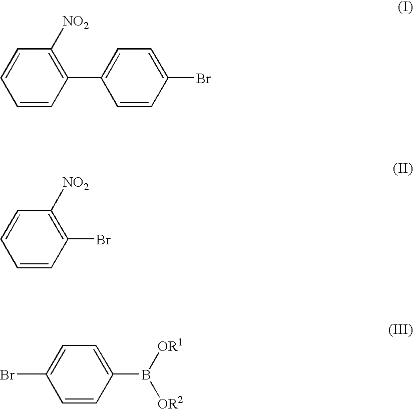

The present invention relates to a novel process for preparing 2-nitro-4′-bromo-biphenyl and its use for preparing 2-nitro- and 2-amino-4′-alkynyl-biphenyl compounds which may be used as intermediates for the manufacture of biphenyl fungicides of the type described in WO 2004/058723. The invention also includes a ‘one-pot’ process for preparing the 2-nitro-4′-alk-ynyl-biphenyl intermediates from 2-nitrobromobenzene and to certain of the intermediates themselves, which are novel compounds.

A process for preparing certain 2-nitrobiphenyls is described in U.S. Pat. No. 6,087,542. Unfortunately the process described is not suitable for preparing 2-nitro-4′-bromo-biphenyl as it leads to the formation of a significant amount of the unwanted bis-coupling product, 2-nitro-4′-(4″-bromophenyl)biphenyl, of the formula (A):

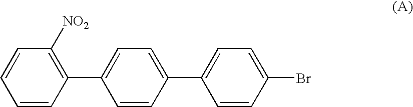

A method for preparing 5-benzyloxy-2-(4-bromophenyl)nitrobenzene from 5-benzyloxy-2-bromonitrobenzene and 4-bromophenylboronic acid by the Suzuki cross-coupling process is described in US 2003/0040538. However, this method uses more than a five-fold molar excess of 4-bromophenylboronic acid to obtain a good yield, which makes it economically and environmentally unsatisfactory.

According to the present invention there is provided a process for the preparation of the compound of the formula (I):

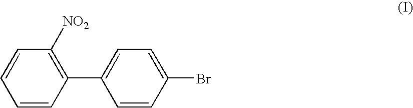

(2-nitro-4′-bromo-biphenyl), which Comprises Reacting the Compound of the Formula (II):

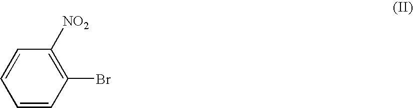

(2-nitrobromobenzene) with a Compound of the General Formula (III):

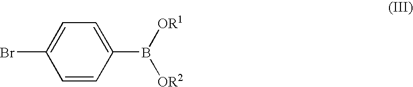

wherein R¹ and R² are H or C₁₋₆ alkyl or R¹ and R² join together to form a C₂₋₃ alkylene group, which is optionally substituted by from 1 to 4 methyl or ethyl groups, or an anhydride of the compound (III), in the presence of a base and a palladium catalyst which is
(a) a palladium (0)- or palladium (II)-triarylphosphine complex optionally in the presence of additional amounts of a triarylphoshine ligand, or
(b) a palladium (II) salt in the presence of a triarylphosphine ligand, or
(c) metallic palladium, optionally deposited on a support, in the presence of triarylphosphine; there being used from 0.9 to 2 moles of compound (III) for each mole of compound (II).

The C₁₋₆ alkyl groups, which R¹ and R² may be, are branched or unbranched alkyl groups containing from 1 to 6 carbon atoms and are, for example, methyl, ethyl, n-propyl, n-butyl, iso-propyl, sec-butyl, iso-butyl, tert-butyl, n-pentyl or n-hexyl. Typically they are methyl, ethyl or iso-propyl. The C₂₋₃ alkylene group, which R¹ and R² may join together to form, is ethylene or propylene, optionally substituted by from 1 to 4 methyl or ethyl groups.

An anhydride of the compound of formula (III) is a product of the combination of two or more equivalents of the compound (III) with elimination of water, containing B—O—B bridges, for example the cyclic anhydride of the formula (IIIa):

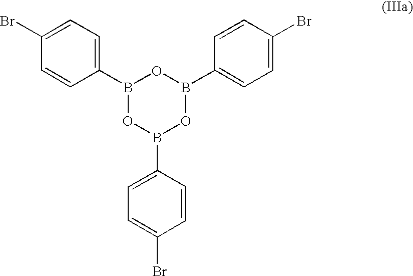

Preferably, 4-bromophenyl boronic acid is employed. If an alkyl ester is used, it is conveniently the dimethyl, diethyl or di-iso-propyl ester.

The amount of compound (III) used in the invention process is from 0.9 to 2 moles for each mole of compound (II), normally from 1.0 to 1.5 moles and preferably about 1.1 moles per mole of compound (II).

The base used may be an organic base, such as a tertiary amine, for example, triethylamine or dimethylcyclohexylamine, but is preferably an alkali metal or alkaline earth metal hydroxide, carbonate, acetate or alkoxide or an alkali metal phosphate or bicarbonate, or mixtures thereof. Particularly suitable are the hydroxides or carbonates of sodium, potassium, lithium, calcium and barium and the phosphates of sodium and potassium.

The amount of base used will depend on the particular base chosen, but for strong inorganic bases such as sodium or potassium hydroxide, it will normally be from 1 to 4, conveniently from 1.5 to 4 and typically about 3 moles per mole of compound (III).

The invention process is carried out in the presence of a palladium catalyst which is either

(a) a palladium (0)- or palladium (II)-triarylphosphine complex optionally in the presence of additional amounts of a triarylphoshine ligand, or
(b) a palladium (II) salt in the presence of a triarylphosphine ligand, or
(c) metallic palladium, optionally deposited on a support, in the presence of triarylphosphine.

Such catalysts are well known to skilled process chemists (see, for example, Angew. Chem. 105 (1993), 1589).

Of the palladium complexes having palladium in the oxidation state 0, tetrakis-(triphenylphosphine)palladium and tetrakis[tri(o-tolyl)phosphine)palladium are particularly suitable. Of the palladium complexes having palladium in the oxidation state plus two, di-(triphenylphosphine)palladium(II) acetate (Pd(O₂CCH₃)₂([C₆H₅]₃P)₂) and di-(triphenyl)-phosphine)palladium(II) chloride (PdCl₂([C₆H₅]₃P)₂) are particularly suitable.

A palladium(II) salt employed in the presence of a triarylphosphine ligand, for example a triphenylphosphine or tri(o-tolyl)phosphine ligand, is suitably palladium(II) acetate or palladium dichloride.

Typically, from 2 to 6 equivalents of the triarylphosphine ligand is complexed with one equivalent of the palladium salt or additionally used with the palladium-triarylphosphine complex.

Metallic palladium is preferably used as a powder or on a support, for example, as palladium on activated carbon, palladium on aluminium oxide, palladium on barium carbonate, palladium on barium sulphate, palladium on calcium carbonate, palladium on aluminium silicates such as montmorillonite and palladium on silic, in each case having a palladium content of 0.5 to 12% by weight. Such supported catalysts may additional contain further doping substances, for example, lead.

When using supported or unsupported metallic palladium, the simultaneous use of a complexed ligand, of the type discussed above, is beneficial, particularly the use of palladium on activated carbon in the presence of triphenylphosphine, tri(o-tolyl)phosphine or other triarylphoshine as complexed ligand, the aryl groups being suitably substituted with 1 to 3 sulphonate groups. Suitably, 2 to 3 equivalents of these ligands are used for each equivalent of palladium metal.

Preferred palladium catalysts are di-(triphenylphosphine)palladium(II) acetate, di-(triphenyl)phosphine)palladium(II) chloride and palladium(II) acetate or palladium(II) chloride in the presence of a triphenylphosphine or tri(o-tolyl)phosphine ligand.

In the invention process, the palladium catalyst is employed in a ratio of from 0.01 to 10 mol %, preferably from 0.05 to 5 and especially from 0.1 to 3 mol %, based on compound (II).

The invention process is carried out in a suitable solvent, either an organic solvent or water, or preferably a mixture of both, in which case the organic solvent is preferably miscible or partially miscible with water. Suitable organic solvents are, for example, ethers such as dimethoxyethane, diethylene glycol dimethyl ether, tetrahydrofuran (THF), dioxane and tert-butyl methyl ether; alcohols such as methanol, ethanol, 1-propanol, 2-butanol, ethylene glycol, 1-butanol, 2-butanol and tert-butanol; ketones such as acetone, ethyl methyl ketone and iso-butyl methyl ketone; and amides such as N,N-dimethylformamide, N,N-dimethyl-acetamide and N-methylpyrrolidone. Mixtures of two or more of these solvents may be used, particularly where one of the solvents is water.

The process may be carried out at a temperature of from 0 to 150° C., normally from ambient (room) temperature to 150° C. Usually, the reaction is carried out at the reflux temperature of the solvent system used.

The reaction time will depend, inter alia, on the scale of the process, the proportion of catalyst and ligand used and the temperature, but will usually take from 1 to 48 hours, for example, from 6 to 24 hours, and typically from 10 to 20 hours.

The process is conveniently carried out by mixing the compounds (II) and (III) in a water miscible organic solvent preferentially but not obligatorily under an inert gas atmosphere, most conveniently argon or nitrogen, adding the base and water, and then adding the palladium catalyst and ligand. However, the order of addition is not critical.

When the reaction is adjudged complete, for example, by gas chromatographic analysis of a sample of the reaction mixture, the crude product may be isolated by removing the palladium catalyst by filtration and freeing it of solvent. It may then be purified by standard laboratory techniques. The product, either in its crude or purified state, is a useful intermediate in, for example, the manufacture of biphenyl fungicides of the type described in WO 2004/058723. In this case it may be reacted with a terminal alkyne, for example, of the formula (IV) defined below, using the well-known Sonogashira procedure to form a compound of the formula (V), as defined below, or reduced using standard reduction conditions to form the compound of the formula (VI), as defined below, and the compound (VI) then reacted with a terminal alkyne using the Sonogashira procedure to form a compound of the formula (VII), as defined below.

Thus, according to one aspect of the present invention, there is provided a process which comprises preparing a compound of the formula (I) as previously described and then reacting the compound of the formula (I) with a compound of the general formula (IV):

H—C≡C—R³ (IV)

wherein R³ is H, C₁₋₆ alkyl [optionally substituted by one or more substituents each independently selected from halogen, hydroxy, C₁₋₄ alkoxy, C₁₋₄ haloalkoxy, C₁₋₄ alkylthio, C₁₋₄ haloalkylthio, C₁₋₄ alkylamino, di-(C₁₋₄)alkylamino, C₁₋₄ alkoxycarbonyl, C₁₋₄ alkylcarbonyloxy, and tri-(C₁₋₄)alkylsilyl)], C₂₋₄ alkenyl [optionally substituted by one or more substituents each independently selected from halogen], C₃₋₇ cycloalkyl [optionally substituted by one or more substituents each independently selected from halogen, C₁₋₄ alkyl and C₁₋₄ haloalkyl] or tri-(C₁₋₄)alkylsilyl;
in the presence of a base, a palladium catalyst as previously defined and a copper (I) salt to form a compound of the general formula (V):

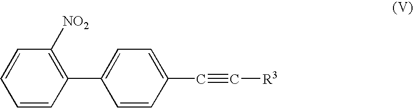

wherein R³ has the meaning given above.

Alkyl groups and the alkyl moieties of alkoxy, alkylthio, alkylamino, etc. are as defined for the alkyl values of R¹ and R² above. Typical values of R³ are H, methyl, iso-propyl, iso-butyl, tert-butyl, cyclopropyl, 1,1-dimethylpropyl, 2,2-dimethylpropyl, hydroxymethyl, hydroxyethyl, 1-hydroxy-1-methylethyl, 1-hydroxy-1-methylpropyl, 1-hydroxymethyl-1-methylethyl, methoxymethyl, 1-methoxy-1-methylethyl, 1-methoxymethyl-1-methylethyl; 1-ethoxy-1-methylethyl, 1-iso-propyloxy-1-methylethyl, 2-methoxy-2-methylbutyl, 2,2,2-tri-fluoroethoxymethyl, 1-methoxycarbonyl-1-methylethyl, 1-methylcarbonyloxy-1-methylethyl, trimethylsilyl and trimethylsilylmethyl.

The base used in this aspect of the invention is preferably an aliphatic or cycloaliphatic primary, secondary or tertiary amine such as piperidine, pyrrolidine, triethylamine, di-isopropyl ethyl amine, diethylamine or n-butylamine. The amount used will normally be from 1 to 4, conveniently from 1.5 to 4 and typically about 3 moles per mole of compound (IV).

The palladium catalyst used may be any catalyst of the type defined in the process for the preparation of compound (I) and in similar amounts.

The copper (I) salt is preferably cuprous iodide. The amount used will normally be from 1 to 6, typically from 1 to 2 equivalents based on the catalyst usage.

The amount of alkyne (IV) used is from 1 to 2 moles, typically from 1.1 to 1.5 moles for each mole of compound (I).

Conveniently the process to form the compound (V) is carried out in a similar solvent system to the one described for the preparation of the compound (I) at ambient or an elevated temperature, for example form 15 to 50° C., typically up to about 40° C., and optionally at a slightly elevated pressure.

It has, however, been found particularly advantageous to prepare the compound (V) in a one-pot reaction sequence by carrying out the Sonogashira process, after the formation of the compound (I), using the compound (I) reaction mixture. A separate, two-stage process would normally necessitate deactivation and removal of the catalyst during work-up and purification and the use of fresh catalyst for the second, Sonogashira stage. This is avoided by the present one-pot process, which not only enables less catalyst to be used overall but results in a better yield over the combined steps and in reduced overall production costs. This is partly the result of the reduced work-up and purification procedures but also, surprisingly, partly due to the apparent reduction in the formation of alkyne oxidative coupling products which are observed when the Sonogashira process is carried out from the isolated compound (I).

Thus, in another aspect of the present invention, there is provided a process as described above wherein the additional step of reacting the compound of the formula (I) with the compound of the general formula (IV) is carried out in the same reaction vessel in which the compound of the formula (I) is prepared, the pH of the reaction mixture in which the compound of the formula (I) is prepared first being reduced to below 9.

In this one-pot process, after completion of the preparation of compound (I), which can be assessed by conventional chromatographic techniques, the crude reaction mixture is typically cooled to room temperature and neutralised by the addition of a dilute acid to a pH below 9, for example below 8, and typically to a pH between 6 and 8. The lower end of the pH range is not critical. However, if the reaction mixture is made too acid, more base than is necessary will need to be added at the subsequent Sonogashira stage. The acid used for neutralisation may be an organic or inorganic acid such as propionic acid or sulphuric acid, or, preferably, acetic acid or hydrochloric acid. The base, cuprous salt and terminal alkyne (IV) are added sequentially and the reaction allowed to proceed, optionally at an elevated temperature and optionally at a slightly elevated pressure, as discussed earlier. Completion of reaction may be adjudged by standard chromatographic techniques.

In yet another aspect of the present invention there is provided a process which comprises preparing a compound of the formula (I) as previously described and then reducing the compound of the formula (I) to form the compound of the formula (VI):

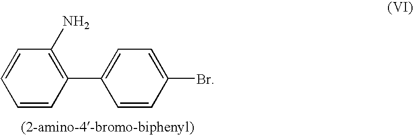

The reduction may be carried out by any suitable well-known literature method for reducing aromatic nitro compounds to anilines. Such methods, which involve inter alia either catalytic or transfer hydrogenation or reduction with metals or metal salts, including methods which allow the presence of additional functional groups like halogens or unsaturated groups, are described in, for example, Houben Weyl: Methoden der organischen Chemie IV/1c, p. 506 et seq., p. 575 et seq. and p 742 et seq.; Houben Weyl: Methoden der organischen Chemie XI/1, p. 394 et seq., which reviews the industrially useful, so-called “Bechamp reduction” with iron; the series, Compendium of Organic Synthetic Methods, Volumes 1-11 (Wiley-Interscience), under the headings “Preparation of amines from nitro compounds”; and Handbook of Catalytic Hydrogenation for Organic Synthesis (S. Nishimura; J. Wiley 2001), Chapter 9.3.

In still yet another aspect of the present invention there is provided a process which comprises preparing a compound of the formula (VI) as described above and then reacting the compound of the formula (VI) with a compound of the general formula (IV):

H—C≡C—R³ (IV)

wherein R³ has the meaning given above, in the presence of a base, a palladium catalyst as previously defined and a copper (I) salt to form a compound of the general formula (VII):

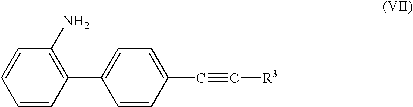

This aspect of the invention may be carried out in a similar fashion, using similar reagents and catalysts in similar proportions, to the Sonogashira process described above for preparing compound (V) from compound (I).

The intermediate chemicals of the general formula (V) are believed to be novel compounds and form still yet a further aspect of the present invention.

Thus, the invention also provides compounds of the general formula (V):

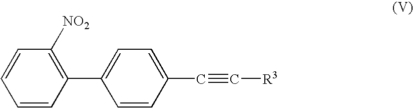

wherein R³ is H, C₁₋₆ alkyl [optionally substituted by one or more substituents each independently selected from halogen, hydroxy, C₁₋₄ alkoxy, C₁₋₄ haloalkoxy, C₁₋₄ alkylthio, C₁₋₄ haloalkylthio, C₁₋₄ alkylamino, di-(C₁₋₄)alkylamino, C₁₋₄ alkoxycarbonyl, C₁₋₄ alkyl-carbonyloxy, and tri-(C₁₋₄)alkylsilyl)], C₂₋₄ alkenyl [optionally substituted by one or more substituents each independently selected from halogen], C₃₋₇ cycloalkyl [optionally substituted by one or more substituents each independently selected from halogen, C₁₋₄ alkyl and C₁₋₄ haloalkyl] or tri-(C₁₋₄)alkylsilyl.

In particular, R³ is H, C₁₋₆ alkyl, C₃₋₆ cycloalkyl, C₁₋₄ haloalkyl, hydroxy(C₁₋₆)alkyl, C₁₋₄ alkoxy(C₁₋₆)alkyl C₁₋₄ haloalkoxy(C₁₋₆)alkyl, C₁₋₄ alkoxycarbonyl(C₁₋₆)alkyl, C₁₋₄ alkyl-carbonyloxy(C₁₋₆)alkyl, tri-C₁₋₄ alkylsilyl or tri-C₁₋₄ alkylsilyl(C₁₋₄)alkyl.

Compounds (V) of especial interest are those where R³ is tert-butyl, 1-methyl-1-meth-oxyethyl or 1-methyl-1-ethoxyethyl.

Illustrative of the compounds of formula (V) are the compounds listed in Table 1 below. The value of R³ is given in the table together with characterising data.

TABLE 1

| Com- | | | ¹H-NMR proton shifts |
|---|---|---|---|
| pound | | | δ (ppm) (CDCl₃) (except |
| No. | R³ | m.p. (° C.) | where otherwise indicated) |
| 1.1 | H | 88-90 | |
| 1.2 | Si(CH₃)₃ | 143-145 | |
| 1.3 | C(CH₃)₃ | 130-132 | |
| 1.4 | CH₃ | 74-76 | |
| 1.5 | cyclopropyl | | |
| 1.6 | CH₂C(CH₃)₃ | | |
| 1.7 | CH(CH₃)₂ | | |
| 1.8 | C(CH₃)₂OH | 86-89 | |
| 1.9 | CH₂OH | 96 | |
| 1.10 | C(CH₃)₂OCH₃ | 85.5-87 | |
| 1.11 | C(CH₃)₂OC₂H₅ | 54-55 | |
| 1.12 | CH₂OCH₃ | | |
| 1.13 | CH₂OCH₂CF₃ | | ¹⁹-F-NMR signal: -74.2 |
| 1.14 | C(CH₃)₂CH₂OCH₃ | | |
| 1.15 | C(CH₃)₂CH₂OH | 126-127 | |
| 1.16 | C(CH₃)₂COOCH₃ | 83.5-84.5 | |
| 1.17 | C(CH₃)₂OCH(CH₃)₂ | | |
| 1.18 | C(CH₃)₂(C₂H₅) | | |
| 1.19 | C(CH₃)(C₂H₅)OCH₃ | | ¹H-NMR: 1.05 (t, 3); 1.48 (s, 3); 1.55 (s, 3); |
| | | | 1.8 (m, 2); 3.4 (s, 3); (7.2-7.9 (m, 8) |
| 1.20 | CH₂CH₂OH | | ¹H-NMR: 1.8 (t, 1; OH); 2.7 (t, 2); 3.8 (q, 2); |
| | | | 7.2-7.9 (m, 8) |
| 1.21 | C(CH₃)(C₂H₅)OH | | ¹H-NMR: 1.08 (2 t; 3); 7.2-7.9 (m, 8) |
| 1.22 | C(CH₃)₂OCOCH₃ | | ¹H-NMR: 1.8 (s, 6); 1.95 (september.; 1) 2.35 (d, 2); |
| | | | 7.2-7.9 (m, 8) |
| 1.23 | CH₂CH(CH₃)₂ | | ¹H-NMR: 1.05 (d, 6); 2.1(s, 3); 7.2-7.9 |
| | | | (m, 8) |
| 1.24 | CH₂Si(CH₃)₃ | 78-79 | |

A nitro compound of the general formula (V) may be reduced to form an amino compound of the general formula (VII) by a suitable reduction process of the type described above for the reduction of the compound of the formula (I) to the compound of the formula (VI). Thus in still yet a further aspect of the present invention there is provided a process which comprises preparing, as described above, a compound of the general formula (V):

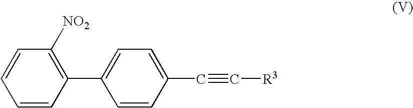

wherein R³ has the meaning given above, and then reducing the compound of the general formula (V) to form the compound of the general formula (VII):

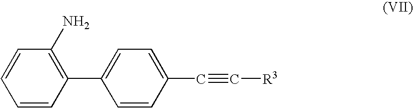

wherein R³ has the same meaning.

Compounds of the general formula (VII) are useful as intermediates for the manufacture of biphenyl fungicides of the type described in WO 2004/058723.

The following non-limiting examples illustrate the invention in more detail.

### EXAMPLE 1

### Preparation of 2-Nitro-4′-bromo-biphenyl

2-Nitrobromobenzene (18.3 g) and 4-bromophenyl-boronic acid (20.0 g) were mixed in a mixture of THF (40 ml) and dimethoxyethane (60 ml) under an atmosphere of nitrogen gas. At room temperature a solution of potassium carbonate (31.3 g) and water (60 ml) was added. The temperature rose to 30° C. and the yellow emulsion was stirred for a few minutes. Tetrakis(triphenylphosphine)palladium (0.63 g) was then added and the resulting mixture was refluxed overnight.

The reaction mixture was cooled to room temperature, filtered and the filtrate was diluted with ethyl acetate. The organic phase was separated, the aqueous phase washed twice with ethyl acetate and the organic phase washed with water and brine and dried over sodium sulfate. After evaporation of the solvent the residue was chromatographed over silica gel (hexane:ethyl acetate 19:1).

Yield: 19.1 g (75.8%) 2-nitro-4′-bromo-biphenyl together with 2.95 g (9.2%) 2-nitro-4′-(4″-bromophenyl)biphenyl of the formula (A):

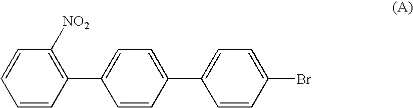

It is a significant advantage of the process according to the invention that the unwanted bis-coupling product (A) is produced in low amounts. Applying the teaching of U.S. Pat. No. 6,087,542 to preparation methods of compounds of formula I leads typically to a significant formation of the unwanted bis-coupling product. In general, ratios in the range of 1:1 of wanted compounds of formula I to unwanted bis-coupling products are observed. In contrast to that, by using the process according to the present invention the amount of the unwanted product is substantially reduced. Typically, ratios in the range of at least 8:1 of compounds of formula I to unwanted bis-coupling products can be obtained, as it is mentioned in example 1.

### EXAMPLE 2

### Preparation of 2-Nitro-4′-(3,3-dimethyl-butyn-1-yl)-biphenyl (Compound 1.3) from 2-nitrobromobenzene by a “One-Pot” Process

2-Nitrobromobenzene (4.6 g) and 4-bromophenyl-boronic acid (5.0 g) were mixed in THF (25 ml) under an atmosphere of nitrogen gas. At room temperature a solution of sodium hydroxide (3.6 g) and water (25 ml) was added. The temperature rose to 33° C. and the yellow emulsion was stirred for a few minutes. Palladium acetate (0.051 g) and triphenylphosphine (0.28 g) were added and the resulting mixture was refluxed for 16 hours. After this time the reaction was completed as judged by gas chromatography. GC analysis showed that less than 1% of biscoupling product (Compound A) had been formed.

The reaction mixture was cooled to room temperature, 2M hydrochloric acid solution was cautiously added until the pH reached 7.5, and triethylamine (50 ml) and cuprous iodide (0.043 g) were added. 3,3-Dimethyl-1-butyne (5.5 ml) was added within one hour. The reaction mixture was then stirred at room temperature for 24 hours and at 40° C. for 15 hours. After cooling the solvents were removed under reduced pressure and the residue was dispersed in tert-butyl methyl ether and water. The organic phase was separated, back-washed with water and brine and the solvent evaporated. The remaining solid (7.7 g) was dissolved in hot hexane and filtered while still hot.

Yield 5.5 g (88.1%; corresponding to an average yield >93% per step); m.p. 130-132° C.

1. A process for the preparation of the compound of the formula (I): 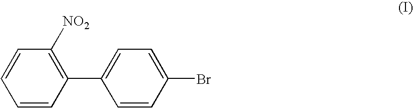
which comprises reacting the compound of the formula (II): 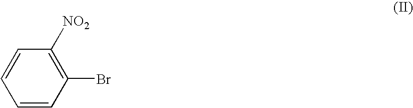
with a compound of the general formula (III): 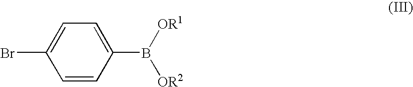
wherein R¹ and R² are H or C₁₋₄ alkyl or R¹ and R² join together to form a C₂₋₃ alkylene group, which is optionally substituted by from 1 to 4 methyl or ethyl groups, or an anhydride of the compound (III), in the presence of a base and of a palladium catalyst which is
(a) a palladium (0)- or palladium (II)-triarylphosphine complex optionally in the presence of additional amounts of a triarylphoshine ligand, or
(b) a palladium (II) salt in the presence of a triarylphosphine ligand, or
(c) metallic palladium, optionally deposited on a support, in the presence of triarylphosphine;
there being used from 0.9 to 2 moles of compound (III) for each mole of compound (II).

2. A process according to claim 1 which comprises the additional step of reacting the compound of the formula (I) with a compound of the general formula (IV):
H—C≡C—R³ (IV)
wherein R³ is H, C₁₋₆ alkyl [optionally substituted by one or more substituents each independently selected from halogen, hydroxy, C₁₋₄ alkoxy, C₁₋₄ haloalkoxy, C₁₋₄ alkylthio, C₁₋₄ haloalkylthio, C₁₋₄ alkylamino, di-(C₁₋₄)-alkylamino, C₁₋₄ alkoxycarbonyl, C₁₋₄ alkylcarbonyloxy, and tri-(C₁₋₄)alkylsilyl)], C₂₋₄ alkenyl [optionally substituted by one or more substituents each independently selected from halogen], C₃₋₇ cycloalkyl [optionally substituted by one or more substituents each independently selected from halogen, C₁₋₄ alkyl and C₁₋₄ haloalkyl] or tri-(C₁₋₄)alkylsilyl;
in the presence of a base, a palladium catalyst as defined in claim 1 and a copper (I) salt to form a compound of the general formula (V): 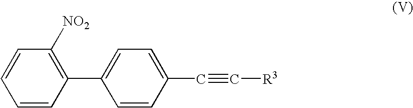
wherein R³ has the meaning given above.

3. A process according to claim 2 wherein the additional step of reacting the compound of the formula (I) with the compound of the general formula (IV) is carried out in the same reaction vessel in which the compound of the formula (I) is prepared, the pH of the reaction mixture in which the compound of the formula (I) is prepared first being reduced to below 9.

4. A process according to claim 1 which comprises the additional step of reducing the compound of the formula (I) to form the compound of the formula (VI):

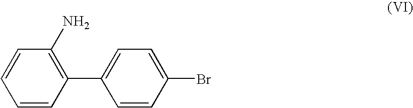

5. A process according to claim 4 which comprises the further step of reacting the compound of the formula (VI) with a compound of the general formula (IV):
H—C≡C—R³ (IV)
wherein R³ has the meaning given in claim 3, in the presence of a base and a copper (I) salt to form a compound of the general formula (VII):

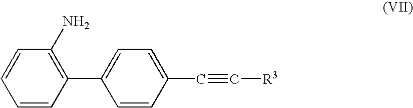

6. A compound of the general formula (V): 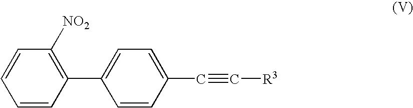
wherein R³ has the meaning given in claim 2.

7. A compound according to claim 6 wherein R³ is H, C₁₋₆ alkyl, C₃₋₆ cycloalkyl, C₁₋₄ haloalkyl, hydroxy(C₁₋₆)alkyl, C₁₋₄ alkoxy(C₁₋₆)alkyl C₁₋₄ haloalkoxy(C₁₋₆)alkyl, C₁₋₄ alkoxycarbonyl(C₁₋₆)alkyl, C₁₋₄ alkylcarbonyloxy(C₁₋₆)alkyl, tri-C₁₋₄ alkylsilyl or tri-C₁₋₄ alkylsilyl(C₁₋₄)alkyl.

8. A compound according to claim 6 wherein R³ is tert-butyl, 1-methyl-1-methoxyethyl or 1-methyl-1-ethoxyethyl.

9. A process according to claim 2 which comprises the further step of reducing the compound of the general formula (V): 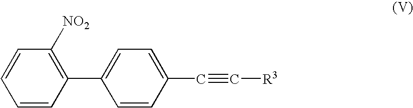
wherein R³ has the meaning given in claim 2, to form a compound of the general formula (VII): 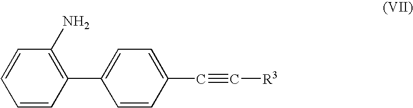
wherein R³ has the same meaning.
